(19) United States
(12) Patent Application Publication
(10) Pub. No.: US 2026/0144904 A1
(43) Pub. Date: May 28, 2026

(54) Mesothelin-Binding Theranostic Fibronectin Type III Tenth Domain (Fn3) Compounds

(71) Applicants: Smith College, Northampton, MA (US); University of Pisa, Pisa (IT)

(72) Inventors: Sarah J. Moore, Amherst, MA (US); Margherita Piccardi, Montepulciano (IT); Roberto Silvestri, Camaiore (IT); Paola Anna Erba, Vergiate (IT); Roberto Giovannoni, Pisa (IT); Stefano Landi, Pisa (IT); Federica Gemignani, Pisa (IT)

(21) Appl. No.: 19/397,408

(22) Filed: Nov. 21, 2025

Related U.S. Application Data
(60) Provisional application No. 63/723,797, filed on Nov. 22, 2024.
(60) Provisional application No. 63/872,799, filed on Aug. 29, 2025.

(51) Int. Cl. A61K 51/08; C07K 14/78; G01N 33/60; G01N 33/68

(52) U.S. Cl. CPC A61K 51/088; C07K 14/78; G01N 33/60; G01N 33/6887; A61K 2121/00; A61K 2123/00; G01N 2333/705; G01N 2333/78

(57) ABSTRACT

Mesothelin-binding theranostic fibronectin type III (Fn3) domain polypeptides conjugated to DOTAGA are described.

### CROSS-REFERENCE TO RELATED APPLICATIONS

The present application claims priority from U.S. Provisional Application No. 63/723,797, filed Nov. 22, 2024, and U.S. Provisional Application No. 63/872,799, filed Aug. 29, 2025. The contents of each of the foregoing applications are hereby incorporated by reference herein in their entirety for all purposes.

### SEQUENCE LISTING

The instant application contains a Sequence Listing which has been submitted electronically in XML format and is hereby incorporated by reference in its entirety. Said XML copy, created on Nov. 20, 2025, is named 4634_1016_SL.xml and is 75,069 bytes in size.

### TECHNICAL FIELD

The present invention relates to mesothelin-binding theranostic fibronectin type III tenth domain (Fn3) compounds, and more particularly to theranostic Fn3 compounds conjugated to DOTAGA and having improved mesothelin binding characteristics.

### BACKGROUND ART

Mesothelin (MSLN) is a tumor biomarker expressed at high levels on the surface of numerous cancers, with extremely limited expression in healthy tissues. MSLN-targeting agents developed for diagnosis and therapy could have a significant impact on the management of MSLN-expressing cancers. Pleural mesothelioma (PM) is a deadly cancer that arises from mesothelial cells lining the pleura, predominantly linked to asbestos exposure. There are currently no effective treatments, and diagnosis occurs in late stages of disease due to the lack of clinical symptoms in early stages.

Due to its overexpression in various tumor types and the preferential location at the membrane site, MSLN represents an attractive molecule for targeted therapies.^{1,2} In addition, MSLN has a role in cancer cell adhesion and metastasis.^{3,4} A recent analysis of more than 12,000 tumors identified various cancer types that might be particularly well suited for anti-MSLN drugs such as ovarian carcinoma, mesothelioma, and adenocarcinomas of the pancreas, lung, stomach, esophagus, and the colorectum.⁵

Among these, pleural mesothelioma (PM) is an aggressive cancer arising from the mesothelial cells lining the pleura and it is predominantly associated with asbestos exposure.⁶ Despite its rarity, PM is a fatal cancer accounting for 26,278 new deaths out of 30,870 new cases in 2020.7 Diagnosis usually occurs 30-50 years after asbestos exposure and in late stages, because clinical signs are not evident in early disease.⁶ Additionally, PM is known to develop resistance to treatments, and surgery is available only for candidate PM cases.^{8,9} Therefore, PM therapy and early diagnosis are still a challenge, leading to poor prognosis with 8-14 months median survival for PM patients.¹⁰

In recent decades, measuring MSLN serum levels and applying MSLN-targeted therapies have been proposed to overcome PM challenges.^{11,12} Despite initial enthusiasm, success has been elusive using antibody-drug conjugates directed at MSLN for PM. A phase II clinical trial with antibody-drug conjugate anetumab ravtansine (BAY 94-9343) targeting MSLN in PM patients was not superior to treatment with chemotherapeutic vinorelbine, although the antibody-drug conjugate did have a clinically manageable safety profile.¹³ Similarly, clinical trials with amatuximab, originally referred to as MORAb-009, have yet to translate into any approved MSLN-targeted therapy.¹⁴⁻¹⁶ Efforts to improve amatuximab and its conjugates to achieve desired clinical outcomes are ongoing.¹⁶⁻¹⁹ While multiple CAR T-cell therapies have been developed targeting MSLN, they have also encountered barriers to success.²⁰⁻²³ Shedding of MSLN from the cell surface has been proposed as one key barrier to overcome for therapies based on antibody and CAR T-cells,^{24,25} which require extended engagement of the therapy at the cancer cell surface to be effective. Recent reports describe MSLN-targeting antibodies and CAR T-cell formats that recognize a domain of MSLN adjacent to the cell membrane and that is not shed from the cell, which may be able to overcome some of the challenges for these classes of therapy.^{26,27}

### SUMMARY OF THE EMBODIMENTS

In accordance with one embodiment of the invention, a theranostic compound comprising (1) a polypeptide comprising a modified human fibronectin type III (Fn3) domain, wherein the polypeptide specifically binds to human mesothelin, and wherein BC, DE, and FG loops of the modified Fn3 domain comprise, respectively, the amino acid sequences set forth in:
(a) SEQ ID NOs: 20, 23, and 25, respectively,
(b) SEQ ID NOs: 20, 23, and 26, respectively,
(c) SEQ ID NOs: 20, 24, and 26, respectively,
(d) SEQ ID NOs: 20, 23, and 27, respectively,
(e) SEQ ID NOs: 20, 23, and 28, respectively,
(f) SEQ ID NOs: 20, 23, and 29, respectively,
(g) SEQ ID NOs: 21, 23, and 30, respectively,
(h) SEQ ID NOs: 22, 23, and 31, respectively, or
(i) SEQ ID NOs: 20, 23, and 32, respectively; and
   (2) 1,4,7,10-tetraazacyclododececane,1-(glutaric acid)-4,7,10-triacetic acid (DOTAGA), wherein the DOTAGA is coupled to the polypeptide.

In some embodiments, non-loop regions the Fn3 domain comprise of amino acid sequences having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to corresponding non-loop regions of SEQ ID NO: 1. In another embodiment, non-loop regions the Fn3 domain comprise of amino acid sequences having 100% identity to corresponding non-loop regions of SEQ ID NO: 1.

In accordance with an embodiment of the invention, a theranostic compound comprising (1) a polypeptide comprising a modified human fibronectin type III (Fn3) domain, wherein (a) non-loop regions of the Fn3 domain comprise of amino acid sequences having at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to corresponding non-loop regions of an Fn3 sequence selected from the group consisting of SEQ ID NOs: 6-19 and 33-36 and (b) BC, DE, and FG loops of the Fn3 domain comprise of amino acid sequences 100% identical to corresponding BC, DE, and FG loops of the Fn3 sequence; and (2) DOTAGA, the DOTAGA being coupled to the polypeptide. In some embodiments, the non-loop regions of the Fn3 domain comprise of amino acid sequences having 100% identity to the corresponding non-loop regions of the Fn3 sequence.

In some embodiments, the polypeptide comprises an amino acid residue having a functional group comprising an amine and the DOTAGA is coupled to the amine group.

In some embodiments, the polypeptide comprises at least one cysteine residue and the DOTAGA is coupled to the at least one cysteine residue. The polypeptide may comprise a single cysteine residue and the DOTAGA is coupled to the single cysteine residue.

In some embodiments a radionuclide is chelated to the DOTAGA. The radionuclide may be copper-64, gallium-68, gallium-69, zirconium-89, lutetium-177, actinium-225, or bismuth-213.

In accordance with one embodiment of the invention, a method of treating a cancer in a subject in need thereof, the method comprising administering to the subject a therapeutically effective amount of the theranostic compound. The cancer may be mesothelioma, a lung adenocarcinoma, a pancreatic adenocarcinoma, a colorectal carcinoma, an ovarian carcinoma, an endometrium carcinoma, a breast carcinoma, cholangiocarcinoma, or a gastric adenocarcinoma.

In accordance with one embodiment of the invention, a method of detecting mesothelin expression by a cell, the method comprising contacting the cell with the theranostic compound; and detecting radiation emitted by the radionuclide of the mesothelin-binding theranostic compound bound to the mesothelin expressed by the cell. The cell may be a cancer cell. The cancer cell may be associated with a cancer selected from the group consisting of mesothelioma, a lung adenocarcinoma, a pancreatic adenocarcinoma, a colorectal carcinoma, an ovarian carcinoma, an endometrium carcinoma, a breast carcinoma, cholangiocarcinoma, and a gastric adenocarcinoma.

In accordance with another embodiment of the invention, a method of detecting mesothelin-expressing tissue in a subject, the method comprising administering the theranostic compound to the subject; and imaging, by an imager configured to detect radiation emitted by the radionuclide of the mesothelin-binding theranostic compound, radiation produced by the radionuclide of the theranostic compound bound to the mesothelin expressed by the tissue. The tissue may be a tumor associated with a cancer. The cancer may be mesothelioma, a lung adenocarcinoma, a pancreatic adenocarcinoma, a colorectal carcinoma, an ovarian carcinoma, an endometrium carcinoma, a breast carcinoma, cholangiocarcinoma, or a gastric adenocarcinoma.

In accordance with one embodiment of the invention, a composition comprising the theranostic compound and a pharmaceutically acceptable carrier.

In accordance with another embodiment of the invention, a kit comprising the theranostic compound and instructions for use.

### BRIEF DESCRIPTION OF THE DRAWINGS

The foregoing features of embodiments will be more readily understood by reference to the following detailed description, taken with reference to the accompanying drawings, in which:

FIG. 1A shows an Fn3 scaffold structure having three solvent exposed loops that form a binding interface for engineering interactions with target molecules (from PDB 1TTG). FIG. 1B is an SDS-PAGE gel showing 5.3.2 was readily expressed and purified from bacterial culture, in accordance with embodiments of the invention.

FIG. 2 is a plot showing a representative binding curve for Fn3 5.3.2 functionalized with DOTAGA-anhydride, in accordance with embodiments of the invention. Data from each of three replicates were fit to a sigmoidal curve. Binding affinity for each replicate was determined as the concentration yielding the half maximum effect, and mean and standard deviation determined as K_{D}=12±11 nM.

FIG. 3 is a graph showing Fn3_{cys} (SEQ ID NO:34)-DOTAGA-⁶⁹Ga bioconjugates melting curves, in accordance with an embodiment of the present disclosure. Melting curves were carried out to explore the Fn3_{cys} stability at different temperatures in a solution at pH 4.5. This assay was performed to select the appropriate conditions for Fn3_{cys}DOTAGA and ⁶⁹Ga coupling. The graph reports melting peaks of Fn3_{cys}-DOTAGA at pH 4.5 without ⁶⁹Ga (red), Fn3_{cys}-DOTAGA with 10× molar excess of ⁶⁹Ga (green), and Fn3_{cys}-DOTAGA with 100× molar excess of ⁶⁹Ga. A solution of sodium acetate 0.1 M and HCl 0.1 M at pH 4.5 was used as a negative control (black). The temperature selected for the subsequent coupling reaction was ˜5° C. below the estimated melting temperature.

FIG. 4A is a binding curve showing Fn3_{cys} 5.3.2 (SEQ ID NO:34) functionalized with maleimide-DOTAGA, using the MSLN-overexpressing line MSTO clone 7, binds MSLN-positive cells with high affinity, in accordance with an aspect of the disclosure. FIG. 4B is a binding curve showing Fn3_{cys} 5.3.2 (SEQ ID NO:34) DOTAGA-⁶⁹Ga, using the MSLN-overexpressing line MSTO clone 7, binds MSLN-positive cells with high affinity, in accordance with an aspect of the disclosure. Data from three replicates were normalized and fit to a sigmoidal curve. The binding affinity for each replicate was determined as the concentration yielding the half maximum effect. For each concentration, mean (dot) and standard deviation (error bars) of the fluorescence intensity from the three replicates are represented in the graph (K_{D}=31±2 nM for Fn3_{cys}-DOTAGA and K_{D}=15 5 nM for Fn3_{cys}-DOTAGA-⁶⁹Ga.).

### DETAILED DESCRIPTION OF SPECIFIC EMBODIMENTS

Disclosed herein are methods for radiolabeling engineered mesothelin-binding Fn3 variants by DOTAGA conjugation and radiometal chelation and demonstrate their use in PM cell lines. These theranostic compounds, which bind mesothelin with high affinity and specificity, are promising candidates for development in preclinical models of PM, towards the goal of better ways to diagnose and treat PM and other MSLN-positive tumors.

Mesothelin-binding Fn3 polypeptides may overcome limitations encountered thus far with antibody-based approaches to treat PM. MSLN-targeting Fn3 5.3.2, disclosed herein, binds MSLN without relying on MSLN glycosylation, which can vary among PM patients. DOTAGA metal chelator was conjugated to MSLN-binding Fn3, and the resulting conjugate maintains binding to MSLN-expressing PM cells. MSLN-binding Fn3-DOTAGA is a promising molecule for chelating with a range of radionuclides with diagnostic and therapeutic relevance, towards applications in molecular imaging and targeted radiotherapy.

Definitions. As used in this description and the accompanying claims, the following terms shall have the meanings indicated, unless the context otherwise requires:

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention

The term “K_{D},” as used herein in the context of a mesothelin-binding Fn3 protein binding to mesothelin means the dissociation equilibrium constant of a particular mesothelin-binding Fn3 protein interaction or the affinity of a mesothelin-binding Fn3 protein for a protein (e.g., mesothelin).

“Polypeptide” as used herein refers to any sequence of two or more amino acids, regardless of length, post-translation modification, or function. “Polypeptide,” “peptide,” and “protein” are used interchangeably herein. Polypeptides can include natural amino acids and non-natural amino acids such as those described in U.S. Pat. No. 6,559,126, incorporated herein by reference. Polypeptides can also be modified in any of a variety of standard chemical ways (e.g., an amino acid can be modified with a protecting group; the carboxy-terminal amino acid can be made into a terminal amide group; the amino-terminal residue can be modified with groups to, e.g., enhance lipophilicity; or the polypeptide can be chemically glycosylated or otherwise modified to increase stability or in vivo half-life). Polypeptide modifications can include the attachment of another structure such as a cyclic compound or other molecule to the polypeptide and can also include polypeptides that contain one or more amino acids in an altered configuration (i.e., R or S; or, L or D). Peptides of the present disclosure are proteins derived from the tenth type III domain of fibronectin that have been modified to bind specifically to human mesothelin.

Percent (%) amino acid sequence identity herein means the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR™) software. Those skilled in the art can readily determine appropriate parameters for measuring alignment, including any algorithms needed to achieve maximal alignment over the full-length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Fn3” and “human fibronectin type III” refers to a human fibronectin type III tenth domain protein. An Fn3 domain is small, monomeric, soluble, and stable. It lacks disulfide bonds and, therefore, is stable under reducing conditions. The overall structure of Fn3 resembles the immunoglobulin fold. Fn3 domains comprise, in order from N-terminus to C-terminus, a beta or beta-like strand, A; a loop, AB; a beta or beta-like strand, B; a loop, BC; a beta or beta-like strand, C; a loop, CD; a beta or beta-like strand, D; a loop, DE; a beta or beta-like strand, E; a loop, EF; a beta or beta-like strand, F; a loop, FG; and a beta or beta-like strand, G. The seven antiparallel 3-strands are arranged as two beta sheets that form a stable core, while creating two “faces” composed of the loops that connect the beta or beta-like strands. Loops AB, CD, and EF are located at one face (“the south pole”) and loops BC, DE, and FG are located on the opposing face (“the north pole”). There are at least 15 different Fn3 modules in human fibronectin, and while the sequence homology between the modules is low, they all share a high similarity in tertiary structure. U.S. Pat. No. 8,933,199 is hereby incorporated by reference for its disclosure of fibronectin type III tenth domain proteins.

As used herein, non-loop regions of a Fn3 domain means the regions of a Fn3 domain other than the BC loop, the DE loop, and the FG loop. Each non-loop region has a discrete and contiguous amino acid sequence. In comparing Fn3 domain sequences, a corresponding non-loop region of a first Fn3 domain means the homologous non-loop region of a second Fn3 domain, e.g., the first non-loop region of the first Fn3 domain corresponds with the first non-loop region of the second Fn3 domain.

“Specifically binds,” “specific binding,” “selective binding, and “selectively binds,” as used interchangeably herein in the context of mesothelin-binding Fn3 proteins refers to mesothelin-binding Fn3 proteins that exhibit affinity for human mesothelin, but do not significantly bind (e.g., less than about 10% binding) to a different polypeptide as measured by a technique available in the art such as, but not limited to, an equilibrium binding assay and/or a competitive binding assay (e.g., competition ELISA, BIACORE assay).

“Mesothelin” refers to human mesothelin.

A “cancer,” as used herein, refers a broad group of various diseases characterized by the uncontrolled growth of abnormal cells, which express mesothelin, in the body. Unregulated cell division and growth divide and grow results in the formation of malignant tumors that invade neighboring tissues and may also metastasize to distant parts of the body through the lymphatic system or bloodstream.

“Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication, condition or biochemical indicia associated with a disease.

“Administration” or “administering,” as used herein refers to introducing a mesothelin-binding theranostic compound described herein into a subject. Any route of administration is suitable, such as intravenous, oral, topical, subcutaneous, peritoneal, intra-arterial, inhalation, vaginal, rectal, nasal, introduction into the cerebrospinal fluid, or instillation into body compartments can be used.

The term “therapeutically effective amount” refers to at least the minimal dose, but less than a toxic dose, of an agent which is necessary to impart a therapeutic benefit to a subject.

The term “pharmaceutically acceptable carrier” means solvents, carrier agents, diluting agents and the like which are used in the administration of pharmaceutical compounds.

The term “therapeutic” as used herein means having a biological effect or combination of biological effects that prevents, inhibits, eliminates, or prevents progression of a disease or other aberrant biological processes in an animal.

The term “diagnostic” as used herein means having the ability to detect, monitor, follow, and/or identify a disease or condition in an animal (including humans) or from a biological sample including but not limited to blood, urine, saliva, sweat and fecal matters.

The term “theranostic” as used herein means having the combined effects of a therapeutic and a diagnostic composition.

The term “radionuclide,” also known as a radioisotope, as used herein refers to an unstable form of a chemical element that releases radiation as it breaks down and becomes more stable (radioactive decay). Radionuclides may be used in imaging tests and in treatment.

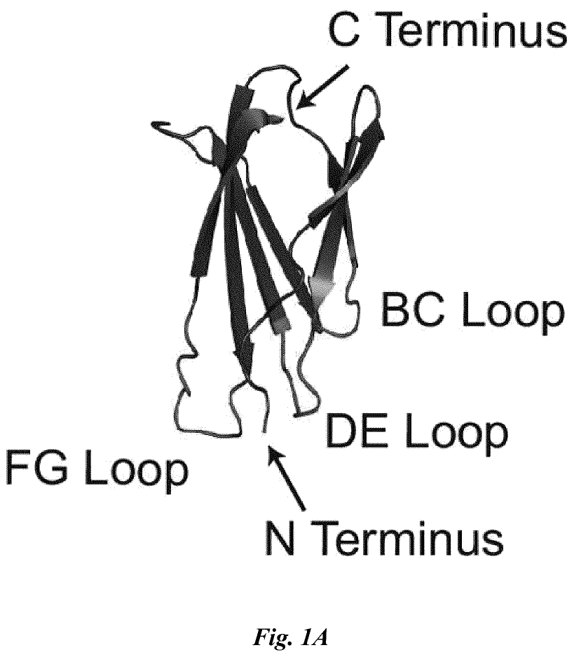

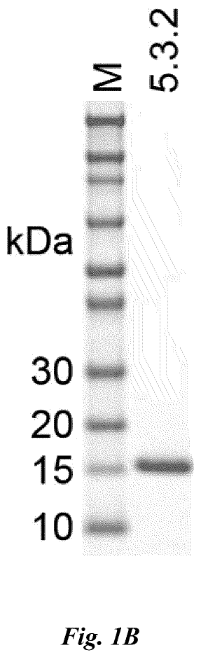

MSLN-binding Fn3 variants identified from an affinity matured fourth and fifth-generation library using yeast surface display were obtained courtesy of A. Sirois and S. Moore. Silvestri, R. et al. “Development of a mesothelin-binding engineered scaffold protein as a theranostic for pleural mesothelioma” (unpublished manuscript). FIG. 1A shows an Fn3 scaffold structure having three solvent exposed loops that form a binding interface for interactions with target molecules. SEQ ID NO:1 is a Fn3 hydrophilic library general amino acid sequence (SEQ ID NO:1), wherein X indicates any amino acid. BC, DE, and FG loops (shown, respectively, as contiguous sequences of any amino acid, i.e., “X”) may vary in length.

Two predominant MSLN-binding Fn3 variants, 5.3.1 (SEQ ID NO:7) and 5.3.2 (SEQ ID NO: 8), in addition to 11 MSLN-binding Fn3 binding variants with low representation (SEQ ID Nos: 9-19) were obtained from the fifth generation library, and one Fn3 variant, 4.4.8 (SEQ ID NO:6) was obtained from the fourth generation library. Id. 5.3.1 and 5.3.2 were reported to exhibit greater binding signals compared to all other variants tested, with 5.3.2 demonstrating the highest affinity to an Fc-MSLN. Id. We therefore pursued Fn3 5.3.2 for additional characterization and bioconjugate modification towards nuclear medicine applications, particularly for PM.

In accordance with some embodiments, a theranostic compound comprising (1) a polypeptide of the present disclosure comprises a modified human fibronectin type III (Fn3) domain, wherein the polypeptide specifically binds to human mesothelin, and wherein BC, DE, and FG loops of the modified Fn3 domain comprise, respectively, the amino acid sequences set forth in:
(a) SEQ ID NOs: 20, 23, and 25, respectively,
(b) SEQ ID NOs: 20, 23, and 26, respectively,
(c) SEQ ID NOs: 20, 24, and 26, respectively,
(d) SEQ ID NOs: 20, 23, and 27, respectively,
(e) SEQ ID NOs: 20, 23, and 28, respectively,
(f) SEQ ID NOs: 20, 23, and 29, respectively,
(g) SEQ ID NOs: 21, 23, and 30, respectively,
(h) SEQ ID NOs: 22, 23, and 31, respectively, or
(i) SEQ ID NOs: 20, 23, and 32, respectively; and
   (2) 1,4,7,10-tetraazacyclododececane,1-(glutaric acid)-4,7,10-triacetic acid (DOTAGA), wherein the DOTAGA is coupled to the polypeptide.

In some embodiments, the BC loop, and/or the DE loop, and/or the FG loop comprises one amino acid substitution, such as a conservative amino acid substitution. In another embodiment, the BC loop, and/or the DE loop, and/or the FG loop comprises one amino acid deletion. In some embodiments, the BC loop, and/or the DE loop, and/or the FG loop comprises one amino acid insertion.

In some embodiments, each non-loop region of the Fn3 domain comprises an amino acid sequence having at least 80, 85, 86, 87, 88, 89, 90, 91, 92, 93, 94, 95, 96, 97, 98, 99, or 100% identity to a corresponding non-loop region of SEQ ID NO: 1.

In some embodiments, each non-loop region of the Fn3 domain consists of an amino acid sequence having at least 80, 85, 86, 87, 88, 89, 90, 91, 92, 93, 94, 95, 96, 97, 98, 99, or 100% identity to a corresponding non-loop region of SEQ ID NO: 1.

In another embodiment, a theranostic compound comprising (1) a polypeptide comprising a modified human fibronectin type III (Fn3) domain, wherein (a) each non-loop region of the Fn3 domain comprises an amino acid sequence having at least 80, 85, 86, 87, 88, 89, 90, 91, 92, 93, 94, 95, 96, 97, 98, 99, or 100% identity to a corresponding non-loop region of an Fn3 sequence selected from the group consisting of SEQ ID NOs: 6-19 and 33-36 and (b) BC, DE, and FG loops of the Fn3 domain comprise of amino acid sequences 100% identical to corresponding BC, DE, and FG loops of the Fn3 sequence; and (2) DOTAGA, the DOTAGA being coupled to the polypeptide.

In another embodiment, a theranostic compound comprising (1) a polypeptide comprising a modified human fibronectin type III (Fn3) domain, wherein (a) each non-loop region of the Fn3 domain consists of an amino acid sequence having at least 80, 85, 86, 87, 88, 89, 90, 91, 92, 93, 94, 95, 96, 97, 98, 99, or 100% identity to a corresponding non-loop region of an Fn3 sequence selected from the group consisting of SEQ ID NOs: 6-19 and 33-36 and (b) BC, DE, and FG loops of the Fn3 domain consist of amino acid sequences 100% identical to corresponding BC, DE, and FG loops of the Fn3 sequence; and (2) DOTAGA, the DOTAGA being coupled to the polypeptide.

In some embodiments, the BC loop, and/or the DE loop, and/or the FG loop comprises one amino acid substitution, such as a conservative amino acid substitution. In another embodiment, the BC loop, and/or the DE loop, and/or the FG loop comprises one amino acid deletion. In some embodiments, the BC loop, and/or the DE loop, and/or the FG loop comprises one amino acid insertion.

In some embodiments, the Fn3 domain comprises an amino acid sequence selected from the group consisting of SEQ ID NOs: 6-19 and 33-36.

In another embodiment, the polypeptide comprises an amino acid sequence selected from the group consisting of SEQ ID NOs: 6-19 and 33-36.

In some embodiments, the Fn3 domain consists of an amino acid sequence selected from the group consisting of SEQ ID NOs: 6-19 and 33-36.

In another embodiment, the polypeptide consists of an amino acid sequence selected from the group consisting of SEQ ID NOs: 6-19 and 33-36.

In yet another embodiment, the polypeptide comprises an amino acid residue having a functional group comprising an amine and the DOTAGA is coupled to the amine group.

In some embodiments, the polypeptide comprises at least one cysteine residue and the DOTAGA is coupled to the at least one cysteine residue. The polypeptide may comprise a single cysteine residue and the DOTAGA is coupled to the single cysteine residue.

In some embodiments of the present disclosure, a radionuclide is chelated to the DOTAGA of the theranostic compound. The radionuclide may be copper-64, gallium-68, gallium-69, zirconium-89, lutetium-177, actinium-225, or bismuth-213.

In some embodiments, the theranostic compound binds to mesothelin with a K_{D} of less than 10 nM, 20 nM, 30 nM, 40 nM, 50 nM, 60 nM, 70 nM, 80 nM, 90 nM, or 100 nM.

In accordance with an embodiment of the present disclosure, a method of treating a cancer in a subject in need thereof is contemplated, the method comprising administering to the subject a therapeutically effective amount of the theranostic compound. The cancer may be mesothelioma, a lung adenocarcinoma, a pancreatic adenocarcinoma, a colorectal carcinoma, an ovarian carcinoma, an endometrium carcinoma, a breast carcinoma, cholangiocarcinoma, or a gastric adenocarcinoma.

In accordance with yet another embodiment of the present disclosure, a method of detecting mesothelin expression by a cell is contemplated, the method comprising contacting the cell with the theranostic compound and detecting radiation emitted by the radionuclide of the mesothelin-binding theranostic compound bound to the mesothelin expressed by the cell. In some embodiments, the cell is a cancer cell. The cancer cell may be associated with a cancer selected from the group consisting of mesothelioma, a lung adenocarcinoma, a pancreatic adenocarcinoma, a colorectal carcinoma, an ovarian carcinoma, an endometrium carcinoma, a breast carcinoma, cholangiocarcinoma, and a gastric adenocarcinoma.

In accordance with another embodiment of the present disclosure, a method of detecting mesothelin-expressing tissue in a subject is contemplated, the method comprising administering the theranostic compound to the subject and imaging, by an imager configured to detect radiation emitted by the radionuclide of the mesothelin-binding theranostic compound, radiation produced by the radionuclide of the theranostic compound bound to the mesothelin expressed by the tissue. The tissue may be a tumor associated with a cancer. The cancer may be mesothelioma, a lung adenocarcinoma, a pancreatic adenocarcinoma, a colorectal carcinoma, an ovarian carcinoma, an endometrium carcinoma, a breast carcinoma, cholangiocarcinoma, or a gastric adenocarcinoma.

In accordance with some embodiments of the present disclosure, a composition comprising the theranostic compound and a pharmaceutically acceptable carrier is contemplated.

In accordance with yet another embodiment of the present disclosure, a kit comprising the theranostic compound and instructions for use is contemplated.

### Example 1: Preparation of Fn3 5.3.2 Functionalized with Radiometal Chelator DOTAGA

A Fn3 5.3.2-DOTAGA bioconjugate was synthesized with 100-, 50-, 5-fold molar excess of DOTAGA-anhydride relative to Fn3 5.3.2 (SEQ ID NO:8), utilizing modifications to previously established protocols.²⁸ Since this step is necessary for further Fn3 radiolabeling, multiple conditions were tested to select the optimal pH and molar ratio of DOTAGA to Fn3 for bioconjugation. The stoichiometry of DOTAGA incorporation per Fn3 molecule was ascertained via ESI mass spectrometry. Despite the propensity for rapid hydrolysis, the cyclic anhydride variant exhibits robust bioconjugation characteristics. When a 5-fold molar excess of DOTAGA-anhydride was used, the conjugation reaction resulted in low efficiency, with ˜40% of Fn3 5.3.2 not functionalized. For 100-, and 50-fold DOTAGA molar excesses, most of the protein was chelated and the analytical data revealed a mass increment consistent with the addition of one, two, or three DOTAGA entities per Fn3 5.3.2 molecule (Table 1). The optimal condition for bioconjugation was selected considering three criteria: a) lowest percentage of unconjugated protein in the analyzed sample; b) promoting the incorporation of one, at most two DOTAGA(s), thus lowering the potential alteration in binding efficiency due to multiple DOTAGAs bound to Fn3; c) fulfilling the other criteria with the lowest DOTAGA molar-excess possible, to facilitate further protein purification from unbound DOTAGA.

TABLE 1
Mass spectrometry of Fn3-DOTAGA conjugates

| | Expected MW | Estimated MW | Composition |
|---|---|---|---|
| Species^{a} | (Da)^{b} | (Da)^{c} | (%)^{d} |
| Fn3 | 12817.28 | 12816.31 | 9.02 |
| Fn3-(1)DOTAGA | 13275.74 | 13275.52 | 31.73 |
| Fn3-(2)DOTAGA | 13734.20 | 13733.72 | 41.08 |
| Fn3-(3)DOTAGA | 14192.66 | 14191.92 | 18.17 |

^{a}Data were retrieved by ESI-TOF mass spectrometry analyzing Fn3 5.3.2 conjugated in triethylammonium bicarbonate pH 7.0 with 50-fold DOTAGA-anhydride molar excess.
^{b}Expected and ^{c}estimated molecular weight (MW) of Fn3, or Fn3 mono-/bi-/tri-functionalized with DOTAGA-anhydride (MW = 458.46 Da).
^{d}The percentage of each species is shown.

Using 50-fold molar excess at pH 7.0 resulted in the most promising condition since more than 90% of the protein was conjugated, with mostly one or two DOTAGAs incorporated for each protein molecule (Table 1).

### Example 2: Fn3 5.3.2 Functionalized with DOTAGA Binds MSLN+ Cells with High Affinity

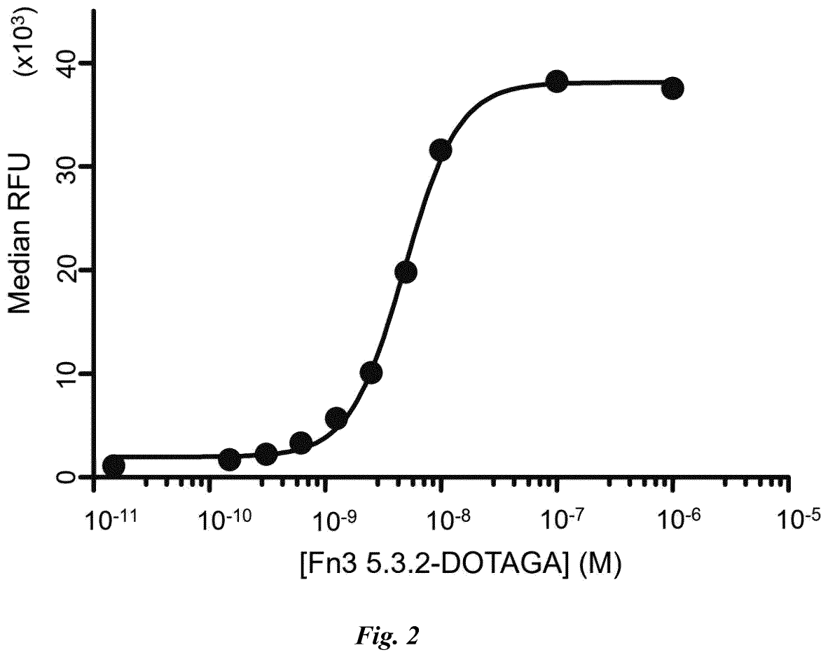

Fn3 5.3.2-DOTAGA conjugates, prepared using 50-fold molar excess at pH 7.0 (purified from excess DOTAGA), were used to evaluate Fn3 5.3.2-DOTAGA binding to MSLN on PM cell lines. Fn3-DOTAGA maintained binding to MSLN with similar affinity as unconjugated Fn3 (K_{D}=12±11 nM, FIG. 2). However, higher variability in the measurement of the K_{D} is observed compared to unconjugated Fn3 5.3.2, likely due to the multiple molecules of DOTAGA attached to Fn3 5.3.2, resulting in a mixture of different species likely characterized by different affinities for MSLN.

The optimal condition for bioconjugations is monolabeling, i.e. 1:1 DOTAGA/antibody stoichiometry, which would ensure more reproducible radiolabeling and facilitate use in in vitro and in vivo models.²⁹⁻³¹ Considering the Fn3 5.3.2 amino acid sequence, five primary amines are potentially available for bioconjugation with DOTAGA-anhydride (2,2′,2″-(10-(2,6-dioxotetrahydro-2H-pyran-3-yl)-1,4,7,10-tetraazacyclododecane-1,4,7-triyl)triacetic acid), including the N-terminus and four lysine residues. This is reflected in the presence, in any reaction condition, of products at several labeling degrees.

Conjugation chemistry for enabling bioconjugation of a Fn3 polypeptide with DOTAGA in a 1:1 stoichiometry is also possible. For example, a single cysteine residue may be introduced into a Fn3 polypeptide, e.g., SEQ ID Nos: 33-36. The single thiol group of the resulting polypeptide may then be reacted with DOTAGA-maleimide (2,2′,2″-(10-(1-carboxy-4-((2-(2,5-dioxo-2,5-dihydro-1H-pyrrol-1-yl)ethyl)amino)-4-oxobutyl)-1,4,7,10-tetraazacyclododecane-1,4,7-triyl)triacetic acid) to achieve a 1:1 stoichiometry.

### Example 3: Fn3_{cys} 5.3.2 Functionalized with Radiometal Chelator DOTAGA and Coupled with ⁶⁹Ga Maintains Binding to MSLN-Positive MSTO Cell Lines

Fn3 5.3.2 bioconjugation with primary amine chemistry resulted in a mixture of different species, with varying degrees of labeling, likely characterized by different affinities for MSLN. Therefore, we further modified Fn3 5.3.2 to enable site-specific thiol-maleimide chemistry. We designed a S97C mutation (Fn3_{cys} 5.3.2) (SEQ ID NO:34), in the C-terminus region of the scaffold, physically opposite the binding loops that recognize with MSLN, avoiding steric hindrance that would likely occur with modifications at the N-terminus. Fn3 5.3.2 does not otherwise have any cysteine residues. In this way, we obtained a unique site for bioconjugation via thiol-maleimide chemistry. A Fn3_{cys}-DOTAGA bioconjugate was synthesized with 25-fold molar excess of maleimide-DOTAGA relative to Fn3_{cys} in PBS 1× and 5 mM TCEP. ESI mass spectrometry analyses showed ˜100% conversion with Fn3:DOTAGA in a 1:1 stoichiometry.

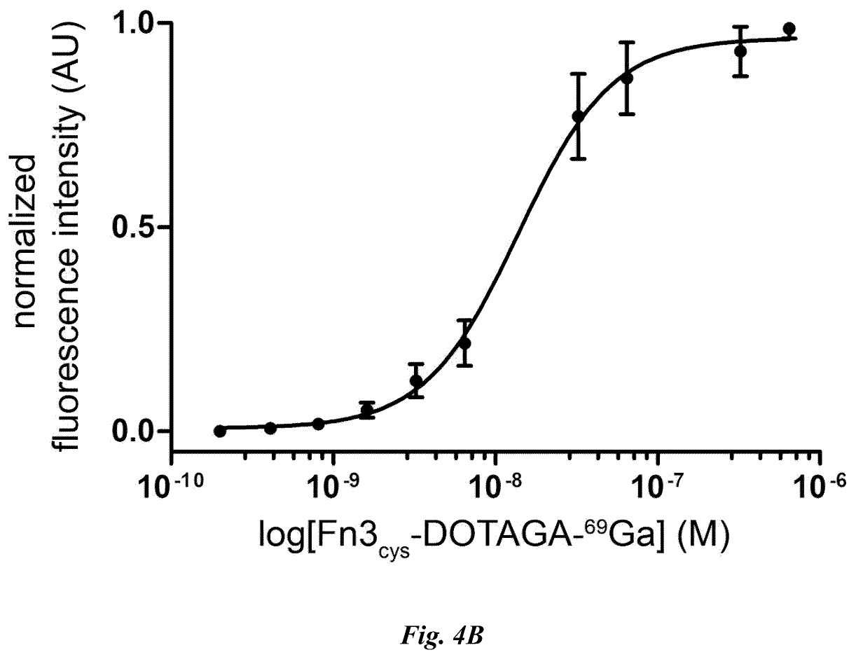

Then, the Fn3_{cys}-DOTAGA conjugates were used to evaluate binding to MSLN on PM cell lines, using the same protocol for equilibrium binding assays that was employed for unconjugated Fn3 5.3.2. Fn3_{cys}-DOTAGA maintained binding to MSLN with similar affinity as unconjugated Fn3 5.3.2 (K_{D}=31±2 nM, FIG. 4A). Thus, S97C substitution and bioconjugation via thiol-maleimide chemistry resulted in an efficient strategy for chelator conjugation.

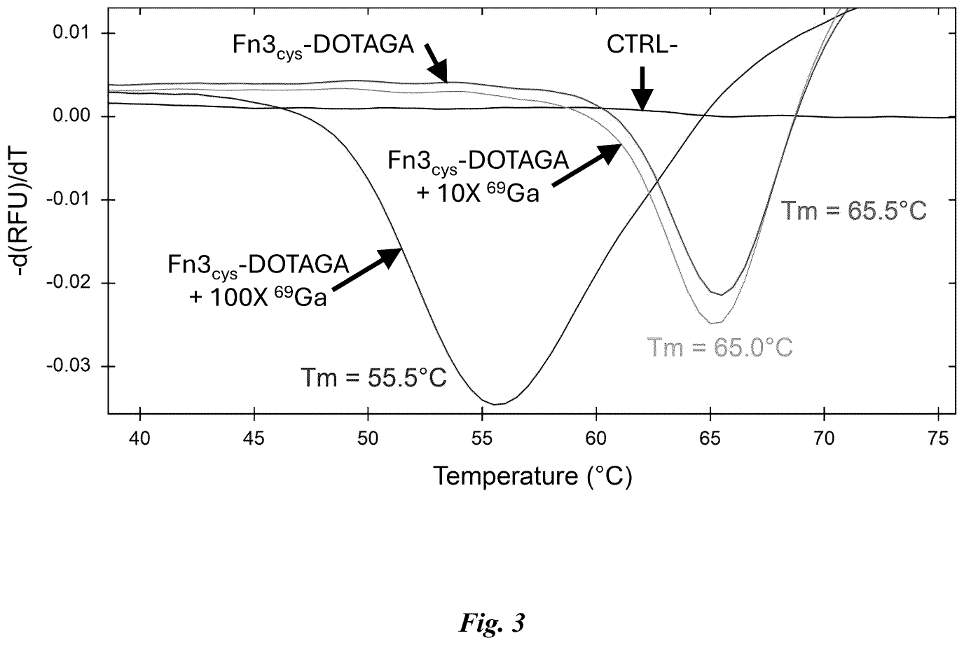

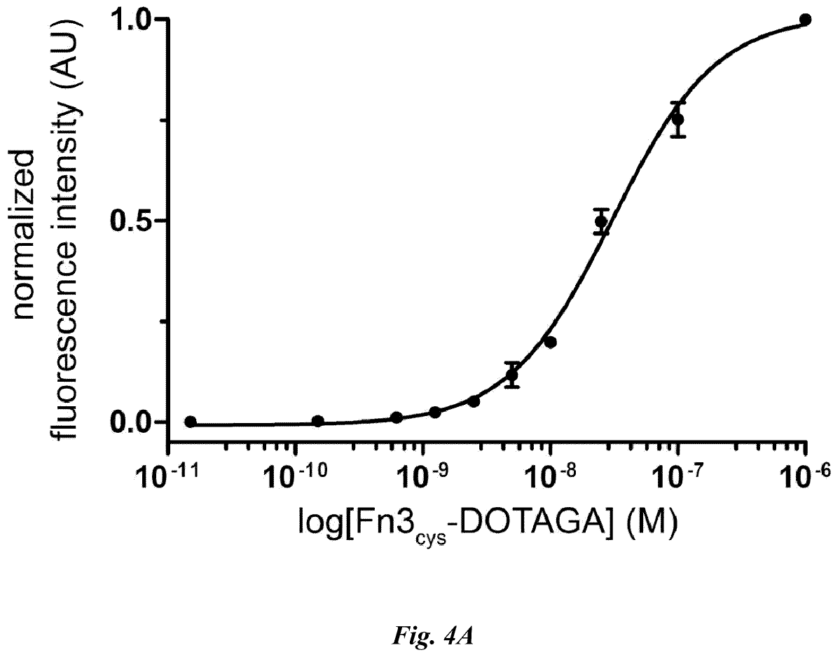

After confirming efficient chelator conjugation and low-nanomolar binding affinity for MSLN, Fn3_{cys}-DOTAGA conjugates were coupled with ⁶⁹Ga. In previous studies, ⁶⁸Ga was employed to study the biodistribution of Fn3 scaffolds in mouse models, demonstrating high specificity and resolution using PET.^{32,33} We decided to use the cold isotope for preliminary evaluation of Fn3_{cys}-DOTAGA-⁶⁹Ga potential for molecular imaging applications in the context of PM. Since coupling with radiometals usually requires low pHs and high temperatures, we evaluated the thermal stability of Fn3_{cys}-DOTAGA at pH 4.5. Using 10- or 100-fold molar excess of ⁶⁹Ga, we observed that the Fn3_{cys}-DOTAGA melting temperature (Tₘ) was ˜65.5° C., which was similar to the Fn3_{cys}-DOTAGA incubated with 10-fold excess of ⁶⁹Ga (Tₘ˜65° C.) (FIG. 3). The Tₘ estimated when using Fn3_{cys}-DOTAGA with 100-fold excess of ⁶⁹Ga decreased by 10° C. (Tₘ˜55° C.) compared to Fn3_{cys}-DOTAGA, suggesting that ⁶⁹Ga concentration used for coupling could affect protein stability at pH 4.5 (FIG. 3).

Following thermal shift assays, the temperature selected for coupling was ˜5° C. below the estimated Tₘ. Therefore, we tested two labeling conditions, using 10-fold or 100-fold molar excess of ⁶⁹Ga, and assessed the coupling efficiency via ESI mass spectrometry, as described for Fn3_{cys}-DOTAGA bioconjugation. When using 10-fold molar excess for 15 min at 60° C. at pH 4.5, 60% of Fn3_{cys}-DOTAGA was coupled with ⁶⁹Ga. Using 100-fold molar excess of ⁶⁹Ga for 15 min at 50° C. at pH 4.5 resulted in 95% final conversion. The Fn3_{cys}-DOTAGA-⁶⁹Ga resulting from this most favorable condition showed high affinity and specificity toward MSLN-positive cells (K_{D}=15±5 nM) (FIG. 4B).

### Materials and Methods

### Fn3 Expression and Purification

MSLN-binding Fn3 variant 5.3.2 and negative control protein Fn3 RDG were prepared as previously described.^{34,35} Briefly, Fn3 genes were cloned into a pET vector with a C-terminal hexahistidine tag (SEQ ID NO: 37) and expressed in BL21(DE3) E. coli. Cultures were grown in LB and induced overnight at 20° C. with 0.5 mM isopropyl-b-D-thiogalactopyranoside (IPTG). Cells were resuspended in lysis buffer (35 mM Na₂HPO₄×dibasic, 15 mM NaH₂PO₄×monobasic, 500 mM NaCl, 5 mM CHAPS, 25 mM imidazole, 5% glycerol) supplemented with an EDTA-free protease inhibitor (Pierce), and lysed by repeated freezing and thawing. Soluble fractions were isolated by centrifugation. Fn3 variants were purified by cobalt affinity chromatography with HisPur cobalt resin (Thermo Fisher). Protein samples were dialyzed into water, lyophilized, reconstituted with 1×PBS to the desired concentration, and analyzed for purity by SDS-PAGE on a BioRad ChemiDoc MP imaging system (see, e.g., FIG. 1).

Generation of Mesothelioma Cell Line with Enhanced MSLN Expression

MSLN-overexpressing clonal populations were established starting from the MSLN-negative cell line MSTO-211H using a pcDNA3.1(+)-based expression vector. The vector was purchased from Twin Helix (MSLN_pcDNA3.1) and harbored the coding sequence for the MSLN transcript variant 1 (accession number: NM_005823.6) and an upstream CMV constitutive promoter. The same vector also carried the NeoR gene, which provided resistance to the G418 antibiotic. MSLN_pcDNA3.1 (5 μg) was transfected into MSTO-211H cells using Lipofectamine 3000 (Invitrogen) following manufacturer instructions. Six days after transfection, the G418 antibiotic was added and maintained in the culture medium at a final concentration of 500 μg/mL to select those cells with a stable expression of the NeoR gene. After 20 days of G418 selection, MSLN expression was evaluated. To this end, 2×10⁶ cells were harvested using a non-enzymatic solution (0.02% EDTA in PBS) and incubated with either PBS (control) or a primary rabbit anti-MSLN antibody (1:50; #196235, Abcam) for 1 h at 4° C. The cells were then washed twice with a solution of PBSA and incubated with either PBS (control) or a secondary PE-conjugated goat anti-rabbit antibody (1:200, #72465, Abcam) for 30 min at 4° C. Following this staining procedure, MSLN expression was evaluated through flow cytometry, and the MSLN-positive cells were seeded at a single-cell density into 96-well plates using a BD FACSJazz cell sorter. The resulting viable clonal populations were re-evaluated for their MSLN levels following a similar staining procedure, and the clones exhibiting stable MSLN overexpression were selected for evaluating Fn3 5.3.2-MSLN binding affinity.

### Cell Lines and Cell Culture

The MSTO-211H cell line was purchased from the American Type Culture Collection (ATCC, #CRL-2081). The cells were cultured in RPMI-1640 (#ECB2000L, Euroclone, S.p.A., Milan, Italy) supplemented with 10% fetal bovine serum (FBS, #10270-106, Gibco™) and 1% penicillin-streptomycin (#ECB3001D, Euroclone, S.p.A., Milan, Italy). Cells were grown at 37° C. in a humidified atmosphere with 5% CO₂.

Equilibrium Binding Assays with Cancer Cell Lines

Wild-type MSTO and MSLN-overexpressing clones (1 and 7) were detached with 0.02% EDTA (#E5134, Sigma-Aldrich) solution. For each sample, 3×10⁵ cells were pelleted at 400 g for 5 min at 4° C.; then, cells were washed with PBSA. Cells were incubated with a range of concentrations (0.015-1000 nM) of Fn3 5.3.2 in a total volume of 300 μl PBSA for 1 hour at 4° C. with rotation. Cells were washed with PBSA and incubated with a mouse anti-His₆ DyLight-488 antibody (“His6” disclosed as SEQ ID NO: 37) (1:50, 20 g/mL, #117512, Abcam) in a total volume of 50 μl for 30 min at 4° C. with rotation and protection from light. MSLN expression was detected by a rabbit anti-MSLN antibody (1:50; 9 g/mL, #196235, Abcam) and a goat anti-rabbit PE conjugate (1:200, 2.5 g/mL, #72465, Abcam). After incubation, cells were washed with PBSA and analyzed using a CytoFLEX Flow Cytometer (Beckman Coulter). Data was fit to a sigmoidal curve using a four parameter logistic (4PL) regression model. Dissociation constants (K_{D}) were calculated as the Fn3 concentration yielding half of the maximum signal for three replicates. Mean and standard deviation for the K_{D} are shown. Following DOTAGA conjugation and ⁶⁹Ga, binding assays with functionalized Fn3 were conducted similarly.

### Primary Amine DOTAGA Conjugation and Conjugate Characterization

First, to enable radiolabeling of Fn3 5.3.2 (SEQ ID NO:8), the protein was functionalized with DOTAGA-anhydride, exploiting the primary amine at the N-terminus of the scaffold. Fn3 5.3.2 bioconjugation was executed with modifications of procedures described by Moreau and colleagues.²⁸ To assess the best condition for bioconjugation, Fn3 was functionalized with DOTAGA-anhydride using three molar ratios chelator:protein (5:1, 50:1, 100:1) at different pHs (7.0, 7.5, 8.0). A volume of 1.5 mL of a 13 mg/mL suspension of DOTAGA-anhydride (Chematech, Dijon, France) in anhydrous chloroform (Carlo Erba, Val de Reuil, France) was pipetted under ultrasonication and aliquoted into 1.5 mL polypropylene microtubes. The chloroform was then evaporated under a gentle flow of nitrogen. Subsequently, DOTAGA-anhydride aliquots were resuspended in anhydrous acetonitrile (35 μg/μl), and 5 (39 nmol), 50 (390 nmol) or 100 (780 nmol) equivalents were added to 100 L of purified Fn3 (1 mg/mL, 0.10 mg, 7.8 nmol, 1 equivalent) in triethylammonium bicarbonate (10 mM, pH 7.0, 7.5, or 8.0). The solution was gently mixed, incubated at RT for 1 h and stored at 4° C. For a first evaluation of the bioconjugate, unbound DOTAGA was not removed. Fn3 5.3.2-DOTAGA conjugation efficiency was evaluated through electrospray ionization (ESI) mass spectrometry (ThermoFisher Scientific Orbitrap Exploris 120). Before injection into the mass spectrometer, conjugated and non-conjugated protein samples were diluted in a solution of 50% acetonitrile, 0.1% formic acid, and ultrapure water.³⁶ Acquisition range was set between 800-3000 m/z, and quantification was performed on the 12+ cluster. The conjugation efficiency was evaluated considering the relative intensity of the m/z peaks of conjugate and of native protein (12817.28 Da). This protocol was applied to determine the percentage of functionalized protein, assuming similar ionization efficiency of the two compounds. Percentage of functionalized protein was estimated for each sample, at different pHs and molar ratios. Then, the best conjugation condition was used to evaluate Fn3 5.3.2-DOTAGA binding capacity for MSLN as described above. FIG. 2 shows a plot showing a representative binding curve for Fn3 5.3.2 functionalized with DOTAGA-anhydride. Data from each of three replicates were fit to a sigmoidal curve. Binding affinity for each replicate was determined as the concentration yielding the half maximum effect, and mean and standard deviation determined as K_{D}=12±11 nM.

The optimal condition for bioconjugations is monolabeling, i.e. 1:1 DOTAGA/antibody stoichiometry, which would ensure more reproducible radiolabeling and facilitate use in in vitro and in vivo models.²⁹⁻³¹ To this end, multiple conditions were tested to find the optimal pH and DOTAGA-molar excess allowing the 1:1 stoichiometry between protein and chelator. However, considering the Fn3 5.3.2 amino acid sequence, five primary amines are potentially available for bioconjugation with DOTAGA-anhydride, including the N-terminus and four lysine residues. This is reflected in the presence, in all reaction conditions tested, of products with a range of degrees of labeling.

Fn3 5.3.2 Further Engineering to Optimize Bioconjugation with DOTAGA

Fn3-based scaffolds do not include any cysteine or disulfide bonds in their structure, allowing the introduction of a unique cysteine for functionalization and radiolabeling.³² Therefore, Fn3 5.3.2 was further modified by replacing serine 97 with cysteine (Fn3_{cys} 5.3.2) (SEQ ID NO:34). In this way, a unique cysteine was available for bioconjugation with maleimide-DOTAGA, via thiol-maleimide chemistry. A gene block with the Fn3_{cys} 5.3.2 nucleotide sequence was designed with the S97C substitution (Eurofins Genomics eurofinsgenomics.eu). After cloning of the Fn3_{cys} 5.3.2 gene into the pET vector and transformation into DH5a E. coli cells (MAX Efficiency™ DH5a Competent Cells, #18258012, Invitrogen), the amino acid substitution was confirmed by Sanger sequencing. Then, Fn3_{cys} 5.3.2 was expressed and purified into BL21(DE) E. coli cells as described above. Buffer exchange into PBS with 5 mM Tris(2-carboxyethyl)phosphine (TCEP, Merck, #75259) and protein concentration were carried out with Amicon® Ultra centrifugal filtration devices (MWCO 10 kDa, #UFC801024, Merck). Purity of protein sample was analysed by SDS-PAGE on a BioRad ChemiDoc MP imaging system.

### Protein Thermal Shift Assays

To assess the stability of Fn3_{cys} at high temperature and pH 4.5 that are used in radiolabeling conditions, a melting curve of the protein was carried out using ProteOrange® (#40210, LumiProbe). Briefly, Fn3_{cys}-DOTAGA (0.2 mg/ml) was reconstituted in a solution at pH 5.2 of HCl 0.1 M and sodium acetate (NaOAc) 0.5 M. Samples were prepared in optically clear PCR tubes (0.2 mL). ⁶⁹GaCl₃ (analog of radioisotope ⁶⁸Ga) was purchased from ThermoFisher Scientific (#444100250), weighted and resuspended in HCl 0.1 M. A molar excess of 10-fold or 100-fold ⁶⁹GaCl₃ with respect to Fn3_{cys}-DOTAGA were added to protein samples to reach a final concentration of 0.1 mg/ml Fn3_{cys}-DOTAGA (2 g in 20 l) and pH 4.5. Fn3_{cys}-DOTAGA reconstituted in NaOAc 0.1 M and HCl 0.1 M (pH 4.5) was used as a control. For each condition, two replicates were carried out. ProteOrange® working solution 20× was added (1 μl) to each sample. To estimate the melting temperature, samples were incubated in CFX96 Touch Real-Time PCR Detection System (BioRad). The thermocycling protocol consisted of temperatures varying from 20° C.-90° C. in increments of 0.5° C. for 10 seconds.

### Synthesis and Characterization of Fn3Cys-DOTAGA-⁶⁹Ga

Considering the goal of working with these molecules in theranostic applications, we coupled Fn3_{cys} (SEQ ID NO:34)-DOTAGA to the cold metal Gallium-69 (⁶⁹Ga), as an alternative to the widely used radioisotope ⁶⁸Ga.^{37,38 69}GaCl₃ was purchased from ThermoFisher Scientific (#444100250), weighted and resuspended in HCl 0.1 M. For the coupling reaction, Fn3_{cys}-DOTAGA 2 mg/ml (1 mg, 78 nmol) was resuspended in 0.5 mL in a solution of NaOAc 0.5 M and HCl 0.1 M (pH 5.2). ⁶⁹GaCl₃ in HCl 0.1 M was added to the protein sample in 10-(780 nmol, 0.5 mL) or 100-fold (7800 nmol, 0.5 mL) molar excess, leading to a labeling solution at pH 4.5. According to the melting temperature estimation (FIG. 3), Fn3_{cys}-DOTAGA samples were incubated at 60° C. (10× ⁶⁹GaCl₃) or 50° C. (100× ⁶⁹GaCl₃) for 15 min with gentle shaking in a thermo-mixer. Fn3_{cys} was incubated in the same condition and was used as a control. Unbound ⁶⁹GaCl₃ was removed using PD-10 desalting columns (#GE17-0851-01, Cytiva), following manufacturer's instructions. Then, the conjugate was lyophilized and stored at −20° C. until use. Fn3_{cys}-DOTAGA-⁶⁹Ga conjugation efficiency was evaluated through ESI mass spectrometry (ThermoFisher Scientific Orbitrap Exploris 120), as described in the previous section. After identifying the best coupling condition, binding affinity of Fn3_{cys}-DOTAGA-⁶⁹Ga toward MSLN was measured via flow cytometry, as described above.

### Abbreviations

DOTAGA—1,4,7,10-tetraazacyclododececane,1-(glutaric acid)-4,7,10-triacetic acid
Fn3—10th domain of fibronectin type III
IPTG—isopropyl-b-D-thiogalactopyranoside
MACS—magnetic activated cell sorting
MSLN—mesothelin
MW—molecular weight
PBS—phosphate buffered saline
PBSA—phosphate buffered saline, with 0.1% (w/v) bovine serum albumin
PE—phycoerythrin
PM—pleural mesothelioma

### REFERENCES

(1) Bera, T. K.; Pastan, I. Mesothelin Is Not Required for Normal Mouse Development or Reproduction. Mol Cell Biol 2000, 20 (8), 2902-2906. doi.org/10.1128/MCB.20.8.2902-2906.2000.
(2) Yeo, D.; Castelletti, L.; van Zandwijk, N.; Rasko, J. E. J. Hitting the Bull's-Eye: Mesothelin's Role as a Biomarker and Therapeutic Target for Malignant Pleural Mesothelioma. Cancers (Basel) 2021, 13 (16), 3932. doi.org/10.3390/cancers13163932.
(3) Rump, A.; Morikawa, Y.; Tanaka, M.; Minami, S.; Umesaki, N.; Takeuchi, M.; Miyajima, A. Binding of Ovarian Cancer Antigen CA125/MUC16 to Mesothelin Mediates Cell Adhesion. J Biol Chem 2004, 279 (10), 9190-9198. doi.org/10.1074/jbc.M312372200.
(4) Gubbels, J. A.; Belisle, J.; Onda, M.; Rancourt, C.; Migneault, M.; Ho, M.; Bera, T. K.; Connor, J.; Sathyanarayana, B. K.; Lee, B.; Pastan, I.; Patankar, M. S. Mesothelin-MUC16 Binding Is a High Affinity, N-Glycan Dependent Interaction That Facilitates Peritoneal Metastasis of Ovarian Tumors. Mol Cancer 2006, 5 (1), 50. doi.org/10.1186/1476-4598-5-50.
(5) Weidemann, S.; Gagelmann, P.; Gorbokon, N.; Lennartz, M.; Menz, A.; Luebke, A. M.; Kluth, M.; Hube-Magg, C.; Blessin, N. C.; Fraune, C.; Möller, K.; Bernreuther, C.; Lebok, P.; Clauditz, T. S.; Jacobsen, F.; Izbicki, J. R.; Jansen, K.; Sauter, G.; Uhlig, R.; Wilczak, W.; Steurer, S.; Minner, S.; Burandt, E.; Krech, R. H.; Dum, D.; Krech, T.; Marx, A. H.; Simon, R. Mesothelin Expression in Human Tumors: A Tissue Microarray Study on 12,679 Tumors. Biomedicines 2021, 9 (4), 397. doi.org/10.3390/biomedicines9040397.
(6) Carbone, M.; Adusumilli, P. S.; Alexander, H. R.; Baas, P.; Bardelli, F.; Bononi, A.; Bueno, R.; Felley-Bosco, E.; Galateau-Salle, F.; Jablons, D.; Mansfield, A. S.; Minaai, M.; de Perrot, M.; Pesavento, P.; Rusch, V.; Severson, D. T.; Taioli, E.; Tsao, A.; Woodard, G.; Yang, H.; Zauderer, M. G.; Pass, H. I. Mesothelioma: Scientific Clues for Prevention, Diagnosis, and Therapy. CA Cancer J Clin 2019, 69 (5), 402-429. doi.org/10.3322/caac.21572.
(7) Sung, H.; Ferlay, J.; Siegel, R. L.; Laversanne, M.; Soerjomataram, I.; Jemal, A.; Bray, F. Global Cancer Statistics 2020: GLOBOCAN Estimates of Incidence and Mortality Worldwide for 36 Cancers in 185 Countries. CA Cancer J Clin 2021, 71 (3), 209-249. doi.org/10.3322/caac.21660.
(8) Bronte, G.; Incorvaia, L.; Rizzo, S.; Passiglia, F.; Galvano, A.; Rizzo, F.; Rolfo, C.; Fanale, D.; Listi, A.; Natoli, C.; Bazan, V.; Russo, A. The Resistance Related to Targeted Therapy in Malignant Pleural Mesothelioma: Why Has Not the Target Been Hit Yet?Crit Rev Oncol Hematol 2016, 107, 20-32. doi.org/10.1016/j.critrevonc.2016.08.011.
(9) Danlos, F.-X.; Texier, M.; Job, B.; Mouraud, S.; Cassard, L.; Baldini, C.; Varga, A.; Yurchenko, A. A.; Rabeau, A.; Champiat, S.; Letourneur, D.; Bredel, D.; Susini, S.; Blum, Y.; Parpaleix, A.; Parlavecchio, C.; Tselikas, L.; Fahrner, J.-E.; Goubet, A.-G.; Rouanne, M.; Rafie, S.; Abbassi, A.; Kasraoui, I.; Breckler, M.; Farhane, S.; Ammari, S.; Laghouati, S.; Gazzah, A.; Lacroix, L.; Besse, B.; Droin, N.; Deloger, M.; Cotteret, S.; Adam, J.; Zitvogel, L.; Nikolaev, S. I.; Chaput, N.; Massard, C.; Soria, J.-C.; Gomez-Roca, C.; Zaleman, G.; Planchard, D.; Marabelle, A. Genomic Instability and Protumoral Inflammation Are Associated with Primary Resistance to Anti-PD-1+Antiangiogenesis in Malignant Pleural Mesothelioma. Cancer Discov 2023, 13 (4), 858-879. doi.org/10.1158/2159-8290.CD-22-0886.
(10) Nicolini, F.; Bocchini, M.; Bronte, G.; Delmonte, A.; Guidoboni, M.; Crin6, L.; Mazza, M. Malignant Pleural Mesothelioma: State-of-the-Art on Current Therapies and Promises for the Future. Front Oncol 2019, 9, 1519. doi.org/10.3389/fonc.2019.01519.
(11) Melaiu, O.; Melissari, E.; Mutti, L.; Bracci, E.; De Santi, C.; Iofrida, C.; Di Russo, M.; Cristaudo, A.; Bonotti, A.; Cipollini, M.; Garritano, S. I.; Foddis, R.; Lucchi, M.; Pellegrini, S.; Gemignani, F.; Landi, S. Expression Status of Candidate Genes in Mesothelioma Tissues and Cell Lines. Mutation Research Fundamental and Molecular Mechanisms of Mutagenesis 2015, 771, 6-12. doi.org/10.1016/j.mrfmmm.2014.11.002.
(12) Borea, F.; Franczak, M. A.; Garcia, M.; Perrino, M.; Cordua, N.; Smolenski, R. T.; Peters, G. J.; Dziadziuszko, R.; Santoro, A.; Zucali, P. A.; Giovannetti, E. Target Therapy in Malignant Pleural Mesothelioma: Hope or Mirage?Int J Mol Sci 2023, 24 (11), 9165. doi.org/10.3390/ijms24119165.
(13) Kindler, H. L.; Novello, S.; Bearz, A.; Ceresoli, G. L.; Aerts, J. G. J. V.; Spicer, J.; Taylor, P.; Nackaerts, K.; Greystoke, A.; Jennens, R.; Calabrò, L.; Burgers, J. A.; Santoro, A.; Cedres, S.; Serwatowski, P.; Ponce, S.; Van Meerbeeck, J. P.; Nowak, A. K.; Blumenschein, G.; Siegel, J. M.; Kasten, L.; Kochert, K.; Walter, A. O.; Childs, B. H.; Elbi, C.; Hassan, R.; Fennell, D. A. Anetumab Ravtansine versus Vinorelbine in Patients with Relapsed, Mesothelin-Positive Malignant Pleural Mesothelioma (ARCS-M): A Randomised, Open-Label Phase 2 Trial. The Lancet Oncology 2022, 23 (4), 540-552. doi.org/10.1016/S1470-2045(22)00061-4.
(14) Hassan, R.; Kindler, H. L.; Jahan, T.; Bazhenova, L.; Reck, M.; Thomas, A.; Pastan, I.; Parno, J.; O'Shannessy, D. J.; Fatato, P.; Maltzman, J. D.; Wallin, B. A. Phase II Clinical Trial of Amatuximab, a Chimeric Antimesothelin Antibody with Pemetrexed and Cisplatin in Advanced Unresectable Pleural Mesothelioma. Clin Cancer Res 2014, 20 (23), 5927-5936. doi.org/10.1158/1078-0432.CCR-14-0804.
(15) Fujisaka, Y.; Kurata, T.; Tanaka, K.; Kudo, T.; Okamoto, K.; Tsurutani, J.; Kaneda, H.; Okamoto, I.; Namiki, M.; Kitamura, C.; Nakagawa, K. Phase I Study of Amatuximab, a Novel Monoclonal Antibody to Mesothelin, in Japanese Patients with Advanced Solid Tumors. Invest New Drugs 2015, 33 (2), 380-388. doi.org/10.1007/s10637-014-0196-0.
(16) Matsuzawa, F.; Kamachi, H.; Mizukami, T.; Einama, T.; Kawamata, F.; Fujii, Y.; Fukai, M.; Kobayashi, N.; Hatanaka, Y.; Taketomi, A. Mesothelin Blockage by Amatuximab Suppresses Cell Invasiveness, Enhances Gemcitabine Sensitivity and Regulates Cancer Cell Sternness in Mesothelin-Positive Pancreatic Cancer Cells. BMC Cancer 2021, 21 (1), 200. doi.org/10.1186/s12885-020-07722-3.
(17) Mizukami, T.; Kamachi, H.; Fujii, Y.; Matsuzawa, F.; Einama, T.; Kawamata, F.; Kobayashi, N.; Hatanaka, Y.; Taketomi, A. The Anti-Mesothelin Monoclonal Antibody Amatuximab Enhances the Anti-Tumor Effect of Gemcitabine against Mesothelin-High Expressing Pancreatic Cancer Cells in a Peritoneal Metastasis Mouse Model. Oncotarget 2018, 9 (73), 33844-33852. doi.org/10.18632/oncotarget.26117.
(18) Nicolaides, N. C.; Schweizer, C.; Somers, E. B.; Wang, W.; Fernando, S.; Ross, E. N.; Grasso, L.; Hassan, R.; Kline, J. B. CA125 Suppresses Amatuximab Immune-Effector Function and Elevated Serum Levels Are Associated with Reduced Clinical Response in First Line Mesothelioma Patients. Cancer Biol Ther 2018, 19 (7), 622-630. doi.org/10.1080/15384047.2018.1449614.
(19) Fujii, Y.; Kamachi, H.; Matsuzawa, F.; Mizukami, T.; Kobayashi, N.; Fukai, M.; Taketomi, A. Early Administration of Amatuximab, a Chimeric High-Affinity Anti-Mesothelin Monoclonal Antibody, Suppresses Liver Metastasis of Mesothelin-Expressing Pancreatic Cancer Cells and Enhances Gemcitabine Sensitivity in a Xenograft Mouse Model. Invest New Drugs 2021, 39 (5), 1256-1266. doi.org/10.1007/s10637-021-01118-1.
(20) Adusumilli, P. S.; Zauderer, M. G.; Riviere, I.; Solomon, S. B.; Rusch, V. W.; O'Cearbhaill, R. E.; Zhu, A.; Cheema, W.; Chintala, N. K.; Halton, E.; Pineda, J.; Perez-Johnston, R.; Tan, K. S.; Daly, B.; Araujo Filho, J. A.; Ngai, D.; McGee, E.; Vincent, A.; Diamonte, C.; Sauter, J. L.; Modi, S.; Sikder, D.; Senechal, B.; Wang, X.; Travis, W. D.; Gonen, M.; Rudin, C. M.; Brentjens, R. J.; Jones, D. R.; Sadelain, M. A Phase I Trial of Regional Mesothelin-Targeted CAR T-Cell Therapy in Patients with Malignant Pleural Disease, in Combination with the Anti-PD-1 Agent Pembrolizumab. Cancer Discov 2021, 11 (11), 2748-2763. doi.org/10.1158/2159-8290.CD-21-0407.
(21) Castelletti, L.; Yeo, D.; van Zandwijk, N.; Rasko, J. E. J. Anti-Mesothelin CAR T Cell Therapy for Malignant Mesothelioma. Biomark Res 2021, 9 (1), 11. doi.org/10.1186/s40364-021-00264-1.
(22) Pang, N.; Shi, J.; Qin, L.; Chen, A.; Tang, Y.; Yang, H.; Huang, Y.; Wu, Q.; Li, X.; He, B.; Li, T.; Liang, B.; Zhang, J.; Cao, B.; Liu, M.; Feng, Y.; Ye, X.; Chen, X.; Wang, L.; Tian, Y.; Li, H.; Li, J.; Hu, H.; He, J.; Hu, Y.; Zhi, C.; Tang, Z.; Gong, Y.; Xu, F.; Xu, L.; Fan, W.; Zhao, M.; Chen, D.; Lian, H.; Yang, L.; Li, P.; Zhang, Z. IL-7 and CCL19-Secreting CAR-T Cell Therapy for Tumors with Positive Glypican-3 or Mesothelin. J Hematol Oncol 2021, 14 (1), 118. doi.org/10.1186/s13045-021-01128-9.
(23) Schoutrop, E.; El-Serafi, I.; Poiret, T.; Zhao, Y.; Gultekin, O.; He, R.; Moyano-Galceran, L.; Carlson, J. W.; Lehti, K.; Hassan, M.; Magalhaes, I.; Mattsson, J. Mesothelin-Specific CAR T Cells Target Ovarian Cancer. Cancer Res 2021, 81 (11), 3022-3035. doi.org/10.1158/0008-5472.CAN-20-2701.
(24) Zhang, Y.; Chertov, O.; Zhang, J.; Hassan, R.; Pastan, I. Cytotoxic Activity of Immunotoxin SS1P Is Modulated by TACE-Dependent Mesothelin Shedding. Cancer Research 2011, 71 (17), 5915-5922. doi.org/10.1158/0008-5472.CAN-11-0466.
(25) Liu, X.; Chan, A.; Tai, C.-H.; Andresson, T.; Pastan, I. Multiple Proteases Are Involved in Mesothelin Shedding by Cancer Cells. Commun Biol 2020, 3 (1), 728. doi.org/10.1038/s42003-020-01464-5.
(26) Liu, X.; Onda, M.; Watson, N.; Hassan, R.; Ho, M.; Bera, T. K.; Wei, J.; Chakraborty, A.; Beers, R.; Zhou, Q.; Shajahan, A.; Azadi, P.; Zhan, J.; Xia, D.; Pastan, I. Highly Active CAR T Cells That Bind to a Juxtamembrane Region of Mesothelin and Are Not Blocked by Shed Mesothelin. Proc. Natl. Acad. Sci. U.S.A. 2022, 119 (19),
(27) Chakraborty, A.; Onda, M.; O'Shea, T.; Wei, J.; Liu, X.; Bera, T. K.; Pastan, I. A Bispecific Antibody That Targets the Membrane-Proximal Region of Mesothelin and Retains High Anticancer Activity in the Presence of Shed Mesothelin. Molecular Cancer Therapeutics 2024, 23 (7), 1021-1030. doi.org/10.1158/1535-7163.MCT-23-0233.
(28) Moreau, M.; Raguin, O.; Vrigneaud, J.-M.; Collin, B.; Bernhard, C.; Tizon, X.; Boschetti, F.; Duchamp, O.; Brunotte, F.; Denat, F. DOTAGA-Trastuzumab. A New Antibody Conjugate Targeting HER2/Neu Antigen for Diagnostic Purposes. Bioconjugate Chem. 2012, 23 (6), 1181-1188. doi.org/10.1021/bc200680x.
(29) Freise, A. C.; Wu, A. M. In Vivo Imaging with Antibodies and Engineered Fragments. Molecular Immunology 2015, 67 (2), 142-152. doi.org/10.1016/j.molimm.2015.04.001.
(30) Tsuchikama, K.; An, Z. Antibody-Drug Conjugates: Recent Advances in Conjugation and Linker Chemistries. Protein Cell 2018, 9 (1), 33-46. doi.org/10.1007/s13238-016-0323-0.
(31) Karczmarczyk, U.; Sawicka, A.; Garnuszek, P.; Maurin, M.; Wojdowska, W. Does the Number of Bifunctional Chelators Conjugated to a mAb Affect the Biological Activity of Its Radio-Labeled Counterpart?Discussion Using the Example of mAb against CD-20 Labeled with ⁹⁰ Y or ¹⁷⁷ Lu. J. Med. Chem. 2022, 65 (9), 6419-6430. doi.org/10.1021/acs.jmedchem.1c02044.
(32) Zhou H, Bao G, Wang Z, Zhang B, Li D, Chen L, Deng X, Yu B, Zhao J, Zhu X (2022) PET imaging of an optimized anti-PD-L1 probe 68Ga-NODAGA-BMS986192 in immunocompetent mice and non-human primates. EJNMMI Research 12:35.
(33) Robu S, Richter A, Gosmann D, Seidl C, Leung D, Hayes W, Cohen D, Morin P, Donnelly D J, Lipovšek D, et al. (2021) Synthesis and Preclinical Evaluation of a 68Ga-Labeled Adnectin, 68Ga-BMS-986192, as a PET Agent for Imaging PD-L1 Expression. Journal of Nuclear Medicine 62:1228-1234.
(34) Sirois, A. R.; Deny, D. A.; Baierl, S. R.; George, K. S.; Moore, S. J. Fn3 Proteins Engineered to Recognize Tumor Biomarker Mesothelin Internalize upon Binding. PLoS ONE 2018, 13 (5), e0197029. doi.org/10.1371/journal.pone.0197029.
(35) Sirois, A. R.; Deny, D. A.; Li, Y.; Fall, Y. D.; Moore, S. J. Engineered Fn3 Protein Has Targeted Therapeutic Effect on Mesothelin-expressing Cancer Cells and Increases Tumor Cell Sensitivity to Chemotherapy. Biotechnology and Bioengineering 2020, 117 (2), 330-341. doi.org/10.1002/bit.27204.
(36) Donnelly, D. P.; Rawlins, C. M.; DeHart, C. J.; Fornelli, L.; Schachner, L. F.; Lin, Z.; Lippens, J. L.; Aluri, K. C.; Sarin, R.; Chen, B.; Lantz, C.; Jung, W.; Johnson, K. R.; Koller, A.; Wolff, J. J.; Campuzano, I. D. G.; Auclair, J. R.; Ivanov, A. R.; Whitelegge, J. P.; Pasa-Tolid, L.; Chamot-Rooke, J.; Danis, P. O.; Smith, L. M.; Tsybin, Y. O.; Loo, J. A.; Ge, Y.; Kelleher, N. L.; Agar, J. N. Best Practices and Benchmarks for Intact Protein Analysis for Top-down Mass Spectrometry. Nat Methods 2019, 16 (7), 587-594. doi.org/10.1038/s41592-019-0457-0.
(37) Nelson BJB, Andersson J D, Wuest F, Spreckelmeyer S (2022) Good practices for 68Ga radiopharmaceutical production. EJNMIMII Radiopharmacy and Chemistry 7:27.
(38) Kleynhans J, Ebenhan T, Sathekge MM (2024) Expanding Role for Gallium-68 PET Imaging in Oncology. Seminars in Nuclear Medicine 54:778-791.

The publications (including patent publications), web sites, company names, books, manuals, treatise, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:
P1. A theranostic compound comprising:
   (1) a polypeptide comprising a modified human fibronectin type III (Fn3) domain, wherein the polypeptide specifically binds to human mesothelin, and wherein BC, DE, and FG loops of the modified Fn3 domain comprise, respectively, the amino acid sequences set forth in:
      (a) SEQ ID NOs: 20, 23, and 25, respectively,
      (b) SEQ ID NOs: 20, 23, and 26, respectively,
      (c) SEQ ID NOs: 20, 24, and 26, respectively,
      (d) SEQ ID NOs: 20, 23, and 27, respectively,
      (e) SEQ ID NOs: 20, 23, and 28, respectively,
      (f) SEQ ID NOs: 20, 23, and 29, respectively,
      (g) SEQ ID NOs: 21, 23, and 30, respectively,
      (h) SEQ ID NOs: 22, 23, and 31, respectively, or
      (i) SEQ ID NOs: 20, 23, and 32, respectively; and
   (2) 1,4,7,10-tetraazacyclododececane,1-(glutaric acid)-4,7,10-triacetic acid (DOTAGA), wherein the DOTAGA is coupled to the polypeptide.
P2. The theranostic compound of potential claim P1, wherein non-loop regions the Fn3 domain comprise of amino acid sequences having at least 80% identity to corresponding non-loop regions of SEQ ID NO: 1.
P3. The theranostic compound of potential claim P1, wherein non-loop regions the Fn3 domain comprise of amino acid sequences having 100% identity to corresponding non-loop regions of SEQ ID NO: 1.
P4. A theranostic compound comprising (1) a polypeptide comprising a modified human fibronectin type III (Fn3) domain, wherein (a) non-loop regions of the Fn3 domain comprise of amino acid sequences having at least 80% identity to corresponding non-loop regions of an Fn3 sequence selected from the group consisting of SEQ ID NOs: 6-19 and 33-36 and (b) BC, DE, and FG loops of the Fn3 domain comprise of amino acid sequences 100% identical to corresponding BC, DE, and FG loops of the Fn3 sequence; and (2) DOTAGA, the DOTAGA being coupled to the polypeptide.
P5. The theranostic compound of potential claim P4, wherein the non-loop regions of the Fn3 domain comprise of amino acid sequences having 100% identity to the corresponding non-loop regions of the Fn3 sequence.
P6. The theranostic compound according to any one of potential claims P1-5, wherein the polypeptide comprises an amino acid residue having a functional group comprising an amine and the DOTAGA is coupled to the amine group.
P7. The theranostic compound according to any one of potential claims P1-5, wherein the polypeptide comprises at least one cysteine residue and the DOTAGA is coupled to the at least one cysteine residue.
P8. The theranostic compound according to potential claim P7, wherein the polypeptide comprises a single cysteine residue and the DOTAGA is coupled to the single cysteine residue.
P9. The theranostic compound according to any one of the preceding potential claims, wherein a radionuclide is chelated to the DOTAGA.
P10. The theranostic compound of potential claim P9, wherein the radionuclide is selected from the group consisting of copper-64, gallium-68, gallium-69, zirconium-89, lutetium-177, actinium-225, and bismuth-213.
P11. A method of treating a cancer in a subject in need thereof, the method comprising administering to the subject a therapeutically effective amount of the theranostic compound according to any one of potential claims P1-10.
P12. The method of potential claim P11, wherein the cancer is selected from the group consisting of mesothelioma, a lung adenocarcinoma, a pancreatic adenocarcinoma, a colorectal carcinoma, an ovarian carcinoma, an endometrium carcinoma, a breast carcinoma, cholangiocarcinoma, and a gastric adenocarcinoma.
P13. A method of detecting mesothelin expression by a cell, the method comprising:
   contacting the cell with the theranostic compound according to any one of potential claims P9-10; and
   detecting radiation emitted by the radionuclide of the mesothelin-binding theranostic compound bound to the mesothelin expressed by the cell.
P14. The method of potential claim P13, wherein the cell is a cancer cell.
P15. The method of potential claim P14, wherein the cancer cell is associated with a cancer selected from the group consisting of mesothelioma, a lung adenocarcinoma, a pancreatic adenocarcinoma, a colorectal carcinoma, an ovarian carcinoma, an endometrium carcinoma, a breast carcinoma, cholangiocarcinoma, and a gastric adenocarcinoma.
P16. A method of detecting mesothelin-expressing tissue in a subject, the method comprising:
   administering the theranostic compound according to any one of potential claims P9-10 to the subject; and
   imaging, by an imager configured to detect radiation emitted by the radionuclide of the mesothelin-binding theranostic compound, radiation produced by the radionuclide of the theranostic compound bound to the mesothelin expressed by the tissue.
P17. The method of potential claim P16, wherein the tissue is a tumor associated with a cancer.
P18. The method of potential claim P17, wherein the cancer is selected from the group consisting of mesothelioma, a lung adenocarcinoma, a pancreatic adenocarcinoma, a colorectal carcinoma, an ovarian carcinoma, an endometrium carcinoma, a breast carcinoma, cholangiocarcinoma, and a gastric adenocarcinoma.
P19. A composition comprising the theranostic compound according to any one of potential claims P1-10 and a pharmaceutically acceptable carrier.
P20. A kit comprising the theranostic compound according to any one of potential claims P1-10 and instructions for use.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A theranostic compound comprising:
(1) a polypeptide comprising a modified human fibronectin type III (Fn3) domain, wherein the polypeptide specifically binds to human mesothelin, and wherein BC, DE, and FG loops of the modified Fn3 domain comprise, respectively, the amino acid sequences set forth in:
(a) SEQ ID NOs: 20, 23, and 25, respectively,
(b) SEQ ID NOs: 20, 23, and 26, respectively,
(c) SEQ ID NOs: 20, 24, and 26, respectively,
(d) SEQ ID NOs: 20, 23, and 27, respectively,
(e) SEQ ID NOs: 20, 23, and 28, respectively,
(f) SEQ ID NOs: 20, 23, and 29, respectively,
(g) SEQ ID NOs: 21, 23, and 30, respectively,
(h) SEQ ID NOs: 22, 23, and 31, respectively, or
(i) SEQ ID NOs: 20, 23, and 32, respectively; and
(2) 1,4,7,10-tetraazacyclododececane,1-(glutaric acid)-4,7,10-triacetic acid (DOTAGA),
wherein the DOTAGA is coupled to the polypeptide.

2. The theranostic compound of claim 1, wherein each non-loop region of the Fn3 domain comprises an amino acid sequence having at least 85% identity to a corresponding non-loop region of SEQ ID NO: 1.

3. The theranostic compound of claim 1, wherein non-loop regions the Fn3 domain comprise of amino acid sequences having 100% identity to corresponding non-loop regions of SEQ ID NO: 1.

4. A theranostic compound comprising (1) a polypeptide comprising a modified human fibronectin type III (Fn3) domain, wherein (a) each non-loop region of the Fn3 domain comprises an amino acid sequence having at least 85% identity to a corresponding non-loop region of an Fn3 sequence selected from the group consisting of SEQ ID NOs: 7-19 and 33-36 and (b) BC, DE, and FG loops of the Fn3 domain comprise of amino acid sequences 100% identical to corresponding BC, DE, and FG loops of the Fn3 sequence; and (2) DOTAGA, the DOTAGA being coupled to the polypeptide.

5. The theranostic compound of claim 4, wherein the non-loop regions of the Fn3 domain comprise of amino acid sequences having 100% identity to the corresponding non-loop regions of the Fn3 sequence.

6. The theranostic compound of claim 1, wherein the polypeptide comprises an amino acid residue having a functional group comprising an amine and the DOTAGA is coupled to the amine group.

7. The theranostic compound of claim 1, wherein the polypeptide comprises at least one cysteine residue and the DOTAGA is coupled to the at least one cysteine residue.

8. The theranostic compound according to claim 7, wherein the polypeptide comprises a single cysteine residue and the DOTAGA is coupled to the single cysteine residue.

9. The theranostic compound of claim 1, wherein a radionuclide is chelated to the DOTAGA.

10. The theranostic compound of claim 9, wherein the radionuclide is selected from the group consisting of copper-64, gallium-68, gallium-69, zirconium-89, lutetium-177, actinium-225, and bismuth-213.

11. A method of treating a cancer in a subject in need thereof, the method comprising administering to the subject a therapeutically effective amount of the theranostic compound of claim 1.

12. The method of claim 11, wherein the cancer is selected from the group consisting of mesothelioma, a lung adenocarcinoma, a pancreatic adenocarcinoma, a colorectal carcinoma, an ovarian carcinoma, an endometrium carcinoma, a breast carcinoma, cholangiocarcinoma, and a gastric adenocarcinoma.

13. A method of detecting mesothelin expression by a cell, the method comprising:
contacting the cell with the theranostic compound of claim 9; and
detecting radiation emitted by the radionuclide of the mesothelin-binding theranostic compound bound to the mesothelin expressed by the cell.

14. The method of claim 13, wherein the cell is a cancer cell.

15. The method of claim 14, wherein the cancer cell is associated with a cancer selected from the group consisting of mesothelioma, a lung adenocarcinoma, a pancreatic adenocarcinoma, a colorectal carcinoma, an ovarian carcinoma, an endometrium carcinoma, a breast carcinoma, cholangiocarcinoma, and a gastric adenocarcinoma.

16. A method of detecting mesothelin-expressing tissue in a subject, the method comprising:
administering the theranostic compound of claim 9 to the subject; and
imaging, by an imager configured to detect radiation emitted by the radionuclide of the mesothelin-binding theranostic compound, radiation produced by the radionuclide of the theranostic compound bound to the mesothelin expressed by the tissue.

17. The method of claim 16, wherein the tissue is a tumor associated with a cancer.

18. The method of claim 17, wherein the cancer is selected from the group consisting of mesothelioma, a lung adenocarcinoma, a pancreatic adenocarcinoma, a colorectal carcinoma, an ovarian carcinoma, an endometrium carcinoma, a breast carcinoma, cholangiocarcinoma, and a gastric adenocarcinoma.

19. A composition comprising the theranostic compound of claim 1 and a pharmaceutically acceptable carrier.

20. A kit comprising the theranostic compound of claim 1 and instructions for use.


SEQUENCE LISTING (37 sequences)

SEQ ID NO: 1 - protein, 101 aa, synthetic construct
  VARIANT 23..31: note = Any amino acid
  VARIANT 52..56: note = Any amino acid
  VARIANT 76..86: note = Any amino acid
  REGION 1..101: note = General Fn3 scaffold
  REGION 23..31: note = BC loop
  REGION 52..56: note = DE loop
  REGION 76..86: note = FG loop
SSDSPRNLEVTNATPNSLTISWXXXXXXXXXYRITYGETGGNSPSQEFTVPXXXXXATISGLKPGQDYTITVYAVXXXXXXXXXXXPISINYRTEIDKPSQ

SEQ ID NO: 2 - protein, 97 aa, synthetic construct
  REGION 1..97: note = anti-mesothelin Fn3 1.4.1
  REGION 23..31: note = BC loop
  REGION 52..56: note = DE loop
  REGION 76..83: note = FG loop
SSDSPRNLEVTNATPNSLTISWDYPRYNSLYYRITYGETGGNSPSQEFTVPRYYSYATISGLNPGQDYTITVYAVSGGSTSNPISINYRTEIDKPSQ

SEQ ID NO: 3 - protein, 98 aa, synthetic construct
  REGION 1..98: note = anti-mesothelin Fn3 2.4.1
  REGION 23..31: note = BC loop
  REGION 52..56: note = DE loop
  REGION 76..83: note = FG loop
SSDSPRNLEVTNATPNSLTISWDYPRYNSLYYRITYGETGGNSPSQEFTVPRYYSYATISGLNPGQDYTITVYAVSRYNHYSNPISINYRTEIDKPSQ

SEQ ID NO: 4 - protein, 98 aa, synthetic construct
  REGION 1..98: note = anti-mesothelin Fn3 3.4.4
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYSYATISGLNPGQDYTITVYAVSRYNHYSNPISINYRTEIDKPSQ

SEQ ID NO: 5 - protein, 98 aa, synthetic construct
  REGION 1..98: note = anti-mesothelin Fn3 3.4.5
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYSYATISGLNSGQDYTITVYAVSRYNHYLNPISINYRTEINKPSQ

SEQ ID NO: 6 - protein, 98 aa, synthetic construct
  REGION 1..98: note = anti-mesothelin Fn3 4.4.8
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYLYATVSGLSPGQDYTITVYAVSRYNHYSNPISINYRTEIDEPSQ

SEQ ID NO: 7 - protein, 98 aa, synthetic construct
  REGION 1..98: note = anti-mesothelin Fn3 5.3.1
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYPNPISINYRTEIDKPSQ

SEQ ID NO: 8 - protein, 98 aa, synthetic construct
  REGION 1..98: note = anti-mesothelin Fn3 5.3.2
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYSNPISINYRTEIDKPSQ

SEQ ID NO: 9 - protein, 98 aa, synthetic construct
  REGION 1..98: note = anti-mesothelin Fn3 5.3.19
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYLYATISGLKPGQDYTITVYAVSRYNHYSNPISINYRTEIDKPSQ

SEQ ID NO: 10 - protein, 98 aa, synthetic construct
  REGION 1..98: note = anti-mesothelin Fn3 5.3.8
  REGION 23..31: note = BC loop
  REGION 52..56: note = DE loop
  REGION 76..83: note = FG loop
SSDSPRNLEVTNATPNFLTISWDYPRYNALYYRITYGETGGNSPSQEFTVPRYYLYATISGLKPGQDYTITVYAVSRYNHYSNPISINYRTEIDKPSQ

SEQ ID NO: 11 - protein, 98 aa, synthetic construct
  REGION 1..98: note = anti-mesothelin Fn3 5.3.9
  REGION 23..31: note = BC loop
  REGION 52..56: note = DE loop
  REGION 76..83: note = FG loop
SSYSPRNLEVTNATPNSLTISWDYPRYNALYYRITYGETGGNSPSQEFTVPRYYLYATISGLKPGQDYTITVYAVSRYNHYPNPISINYRTEIDKPSQ

SEQ ID NO: 12 - protein, 98 aa, synthetic construct
  REGION 1..98: note = anti-mesothelin Fn3 5.3.11
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYSHATVSGLNPGQDYTITVYVVSRYNHYPNPVSINYRTEIGKPSQ

SEQ ID NO: 13 - protein, 98 aa, synthetic construct
  REGION 1..98: note = anti-mesothelin Fn3 5.3.12
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYPSPISINYRTEIDKPSQ

SEQ ID NO: 14 - protein, 98 aa, synthetic construct
  REGION 1..98: note = anti-mesothelin Fn3 5.3.17
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYSSPISINYRTEIDKPSQ

SEQ ID NO: 15 - protein, 98 aa, synthetic construct
  REGION 1..98: note = anti-mesothelin Fn3 5.3.18
  REGION 23..31: note = BC loop
  REGION 52..56: note = DE loop
  REGION 76..83: note = FG loop
SSYSPRNLEVTDATPNSLTISWDYPRYNALYYRITYGETGGNSPSQEFTVPRYYLYATISGLKPGQDYTITVYAVSRYNHYSSPISINYRTEIDKPSQ

SEQ ID NO: 16 - protein, 98 aa, synthetic construct
  REGION 1..98: note = anti-mesothelin Fn3 5.3.3
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHHSNPISINYRTEIDKPSQ

SEQ ID NO: 17 - protein, 98 aa, synthetic construct
  REGION 1..98: note = anti-mesothelin Fn3 5.3.5
  REGION 23..31: note = BC loop
  REGION 52..56: note = DE loop
  REGION 76..83: note = FG loop
SSDSPRNLEVTNATPNSLTISWDYPRYNALHYRITYGETGGNSPSQEFTVPRYYLYATISGLKPGQDYTITVYAVSRYSHYSNPISINYRTEIDKPSQ

SEQ ID NO: 18 - protein, 98 aa, synthetic construct
  REGION 1..98: note = anti-mesothelin Fn3 5.3.7
  REGION 23..31: note = BC loop
  REGION 52..56: note = DE loop
  REGION 76..83: note = FG loop
SSDSPRNLEVTNATPNSLTISWDYPRYNARYYRITYGETGGNSPSQEFTVPRYYLYATISGLKPGQDYTITVYAVSRYTNYSNPISINYRTEIDKPSQ

SEQ ID NO: 19 - protein, 98 aa, synthetic construct
  REGION 1..98: note = anti-mesothelin Fn3 5.3.15
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNGVSNPISINYRTEIDKPSQ

SEQ ID NO: 20 - protein, 9 aa, synthetic construct
  REGION 1..9: note = BC loop
DYPRYNALY

SEQ ID NO: 21 - protein, 9 aa, synthetic construct
  REGION 1..9: note = BC loop
DYPRYNALH

SEQ ID NO: 22 - protein, 9 aa, synthetic construct
  REGION 1..9: note = BC loop
DYPRYNARY

SEQ ID NO: 23 - protein, 5 aa, synthetic construct
  REGION 1..5: note = DE loop
RYYLY

SEQ ID NO: 24 - protein, 5 aa, synthetic construct
  REGION 1..5: note = DE loop
RYYSH

SEQ ID NO: 25 - protein, 8 aa, synthetic construct
  REGION 1..8: note = FG loop
SRYNHYSN

SEQ ID NO: 26 - protein, 8 aa, synthetic construct
  REGION 1..8: note = FG loop
SRYNHYPN

SEQ ID NO: 27 - protein, 8 aa, synthetic construct
  REGION 1..8: note = FG loop
SRYNHYPS

SEQ ID NO: 28 - protein, 8 aa, synthetic construct
  REGION 1..8: note = FG loop
SRYNHYSS

SEQ ID NO: 29 - protein, 8 aa, synthetic construct
  REGION 1..8: note = FG loop
SRYNHHSN

SEQ ID NO: 30 - protein, 8 aa, synthetic construct
  REGION 1..8: note = FG loop
SRYSHYSN

SEQ ID NO: 31 - protein, 8 aa, synthetic construct
  REGION 1..8: note = FG loop
SRYTNYSN

SEQ ID NO: 32 - protein, 8 aa, synthetic construct
  REGION 1..8: note = FG loop
SRYNGVSN

SEQ ID NO: 33 - protein, 93 aa, synthetic construct
  REGION 1..93: note = anti-mesothelin Fn3 5.3.2, with single cysteine, version 1
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYSNPISINYRTPC

SEQ ID NO: 34 - protein, 98 aa, synthetic construct
  REGION 1..98: note = anti-mesothelin Fn3 5.3.2, with single cysteine, version 2
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYSNPISINYRTEIDKPCQ

SEQ ID NO: 35 - protein, 98 aa, synthetic construct
  REGION 1..98: note = anti-mesothelin Fn3 5.3.2, with single cysteine, version 3
  REGION 23..31: note = BC loop
  REGION 52..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYSNPISINYRTEIDCPSQ

SEQ ID NO: 36 - protein, 107 aa, synthetic construct
  REGION 1..107: note = anti-mesothelin Fn3 5.3.2, with single cysteine, version 4
  REGION 23..31: note = BC loop
  REGION 51..56: note = DE loop
  REGION 76..83: note = FG loop
SSDSPRNLEVTNATPNSLTISWDYPRYNALYYRITYGETGGNSPSQEFTVPRYYLYATISGLKPGQDYTITVYAVSRYNHYSNPISINYRTEIDKPSQGSHHHHHHC

SEQ ID NO: 37 - protein, 6 aa, synthetic construct
HHHHHH